(19) United States
(12) Patent Application Publication
(10) Pub. No.: US 2022/0280692 A1
(43) Pub. Date: Sep. 8, 2022

(54) SURFACE HARDENING FOR A DENTAL IMPLANT

(71) Applicant: ELOS MEDTECH PINOL A/S, Gørløse (DK)

(72) Inventors: Thomas Lundin CHRISTIANSEN, Frederikssund (DK); Morten Stendahl JELLESEN, Søborg (DK); Marcel A.J. SOMERS, Billund (DK); Andreas Frederik Kielsholm KÖRKEL, Græsted (DK)

(21) Appl. No.: 17/631,851

(22) PCT Filed: Aug. 21, 2020
(86) PCT No.: PCT/EP2020/073547

§ 371 Date: Jan. 31, 2022

(30) Foreign Application Priority Data
Aug. 23, 2019 (EP) 19193360.5

(51) Int. Cl. A61L 27/30; C23C 28/04; C23C 8/16; C23C 8/34

(52) U.S. Cl. CPC A61L 27/30; C23C 28/042; C23C 8/16; C23C 8/34; A61L 2400/18; A61L 2430/12

(57) ABSTRACT

The present invention relates to a Group IV metal or alloy component having a protective oxide surface layer, the Group IV metal or alloy component having a core hardness, a diffusion zone having oxygen in solid solution in the range of a level providing a hardness of 120% of the hardness of the material core to the saturation level of the Group IV metal or alloy, and a Group IV metal oxide layer at the surface of the component, the diffusion zone being between the Group IV metal oxide layer and the material core. In another aspect, the invention relates to a method of producing a protective oxide surface layer on a Group IV metal or alloy comprising: providing a workpiece of a Group IV metal or alloy, oxidising the Group IV metal or alloy in a first and a second oxidation step.

### FIELD OF THE INVENTION

The present invention relates to hardening of Group IV metals or alloys. Specifically, a method of producing a protective oxide surface layer on a Group IV metal or alloy is provided as well as a hardened Group IV metal or alloy component. The method and the component are useful for implants, in particular dental implants.

### PRIOR ART

Titanium is a light weight metal with a tensile strength comparable to stainless steel, which naturally reacts with oxygen to form a titanium oxide layer on the surface that provides corrosion resistance. These characteristics make titanium highly attractive in many fields, such as aerospace, military and for industrial processes, and moreover since titanium is biocompatible it is also relevant for medical uses, e.g. as implants. The naturally forming layer of titanium oxide is thin, e.g. in nanometer scale, and for certain applications it can be desirable to increase the thickness of the oxide layer. Several examples of hardening titanium are known from the prior art with the aim of controlling the oxidation.

US 2003/041922 discloses a method of strengthening a titanium alloy to improve wear resistance. The method comprising heating the titanium alloy in an atmosphere of CO₂ at 600 to 900° C. to diffuse C and O atoms into the titanium alloy, but without forming titanium oxide. A preferred temperature range is 800 to 850° C., and in particular at temperatures above 900° C., titanium oxides are formed. The reaction time may be 0.5 to 50 hours.

US 2003/118858 discloses a process for surface treatment of titanium tableware. In the process, a mixed gas containing nitrogen as a main component and an oxygen component is used to diffuse nitrogen and oxygen inside the titanium at a temperature of 700 to 800° C. under reduced pressure. The mixed gas may comprise nitrogen gas and carbon dioxide gas or carbon monoxide gas. The treating temperature in this step is important in order to avoid forming a compound by the reaction of nitrogen and the oxygen component with the titanium, although the tableware may also be coated with a hard coating film, e.g. a nitride, a carbide, an oxide, a nitrido-carbide or a nitrido-carbido-oxide of titanium. The hard coating film may be provided using ion plating.

JPH059702 addresses the problem of curing titanium by surface oxidation where a white and peelable scale layer is formed on the outermost layer, and where there is a problem in terms of blackness and surface stability. Thus, a method is disclosed capable of forming a black surface hardened layer having sufficient stability at low cost. By treating a titanium surface with CO₂ gas, C and O diffuse to a high concentration toward the inside from the titanium surface layer, and a strong hardened layer in which both elements C and O are in solid solution. The heat treatment is performed at 500 to 950° C.

U.S. Pat. No. 4,478,648 discloses a method for producing protective oxide surface layers on a titanium alloy component. In the method, the surface of the object is treated at a temperature between 500 and 900° C. in the presence of a gaseous mixture including water vapour or carbon dioxide as an oxidising agent in an inert carrier gas. The oxidation potential may be adjusted by varying the partial pressure of the oxidising agent, e.g. the partial pressure of CO₂ may be less than 50 mbar. A duration of 4 hours of oxidation using 20 mbar water vapour in argon at 800° C. provided a dense Ti₂AlO₃ layer 10 to 15 μm thick on TiAl16V4.

JPH0797676 discloses a method of surface treatment of a titanium bolt or nut. In the method, CO₂ or nitrogen gas provide oxygen and carbon solid solution hardening of the titanium material, and N₂ reacts with titanium to form a dense nitrided layer.

WO 2017/207794 explains how a component of a Group IV metal, e.g. a titanium alloy, can be oxidised using a mixture of CO and CO₂, or H₂O and H₂, where the partial pressure of O₂ (pO₂) can be determined from the ratio between CO and CO₂, or H₂O and H₂, respectively. Specifically, this approach allows a Magnéli phase to be formed on titanium in its pure form without the presence of metal oxides, e.g. rutile or TiO₂, on or in the metal. Formation of a Magnéli phase is exemplified, and by decreasing the partial pressure of CO (compared to CO₂) the amount of Ti₄O₇ increases in the Magnéli phases.

US 2005/234561 discloses a wear-resistant titanium alloy orthopedic device and a method of forming a wear-resistant titanium alloy orthopedic device by deeply diffusing oxygen into the titanium alloy device. A method of hardening a device formed of titanium by deeply diffusing oxygen into the titanium alloy device is also disclosed.

GB2159542 discloses a method for producing oxidic protective layers by the treatment of metallic surface in an oxidizing atmosphere in which the O₂ pressure of the treatment medium, governing the oxidation potential as it does, is continuously varied during the oxidation process to control the nucleation and growth rate of the oxide being produced. This is thought to result in a dense, isotropic structure of the resultant oxide layer with a minimum of defect concentration.

U.S. Pat. No. 6,328,819 discloses a method for heat treatment of metal workpieces to obtain a substantially uniform nitride layer, even in workpieces comprising high alloy iron articles.

Thus, the prior art fails to provide a white robust surface layer of titanium oxide, and the present invention aims to address this problem.

### DISCLOSURE OF THE INVENTION

In a first aspect, the present invention relates to a Group IV metal or alloy component comprising a material core having a core hardness, a diffusion zone having oxygen in solid solution in the range of a level providing a hardness of 120% of the hardness of the material core to the saturation level of the Group IV metal or alloy, and a Group IV metal oxide layer at the surface of the component, the diffusion zone being between the Group IV metal oxide layer and the material core,

wherein the Group IV metal oxide layer has a thickness in the range of 5 μm to 100 μm, a carbon content and a cross-sectional hardness of at least 650 HV_{0.005}. The Group IV metal oxide layer can be considered to be a protective oxide surface layer. Thus, the invention provides a Group IV metal or alloy component having a protective oxide surface layer.

In another aspect, the present invention relates to a method of producing a protective oxide surface layer on a Group IV metal or alloy, the method comprising the steps of:

providing a workpiece of a Group IV metal or alloy,

oxidising the Group IV metal or alloy in a first oxidation step at a temperature in the range of 500° C. to 900° C. using a carbon containing gaseous species having a first oxidising potential to provide an intermediary Group IV metal oxide,

oxidising the intermediary Group IV metal oxide in a second oxidation step at a temperature in the range of 300° C. to 900° C. using a second gaseous species having a second oxidising potential to provide the protective oxide surface layer, which second oxidising potential is higher than the first oxidising potential.

The Group IV metal or alloy component of the first aspect of the invention is obtainable in the method of the second aspect of the invention.

The invention relates to a Group IV metal or alloy component. In the context of the invention, the component may be made from a Group IV metal or an alloy containing a Group IV metal at 50% (a/a) or more. The component may also contain other materials, e.g. a core of another material. Any Group IV metal is appropriate for both aspects of the invention. In specific embodiments the Group IV metal or alloy is selected from the list of titanium, titanium alloys, zirconium and zirconium alloys. In the context of the invention the component may consist of the titanium alloy, or a Group IV metal, or it may comprise other materials. For example, the component may have a core of another material, a polymer, glass, ceramic or another metal, and an outer layer of the titanium alloy. The outer layer need not completely cover the outer surface of the component. The component may for example be prepared from additive manufacturing or 3D printing prior to be treated in the methods of the invention. The protective oxide surface layer produced in the method of the invention and also the Group IV metal or alloy component are particularly suited for dental implants. An appropriate Group IV alloy for dental implants is disclosed in U.S. Pat. No. 8,168,012, which is hereby incorporated by reference. The Group IV alloy of U.S. Pat. No. 8,168,012 is a binary single phase titanium-zirconium alloy with a zirconium content of 9.9 wt % to 19.9 wt % and 0.1% to 0.3% by weight of oxygen.

The method of the invention can be considered to represent a two-step oxidation of the Group IV metal or alloy. The first oxidation step uses a carbon containing gaseous species and the second oxidation step uses a second gaseous species so that the first oxidation step oxidises the Group IV metal or alloy and the second oxidation step oxidises the intermediary Group IV metal oxide formed in the first oxidation step. The present inventors have now surprisingly found that by using a carbon containing gaseous species with a low oxidation potential, such as CO₂ or a mixture of CO₂ and CO, e.g. at a ratio of CO₂ to CO and CO₂ in the range of 0.4 to 0.9 or higher, a dense and stable layer of Group IV metal oxide, i.e. the intermediary Group IV metal oxide, is formed on the surface of the Group IV metal or alloy, and that the stability of the intermediary Group IV metal oxide allows that the intermediary Group IV metal oxide is oxidised further in the second oxidation step using a second gaseous species having a higher oxidising potential than the carbon containing gaseous species. In contrast, if the second gaseous species having the high oxidising potential is used directly with the Group IV metal or alloy, unstable layers of Group IV metal oxides are formed on the surface of the Group IV metal. For example, direct oxidation of pure titanium with N₂O, O₂ or air, e.g. at ambient pressure, may result in stratified titanium oxide layers that readily scale off the titanium metal. An exemplary stratified oxide layer is depicted in FIG. 16. Without being bound by theory, the present inventors believe that the carbon containing gaseous species will introduce an amount of carbon into the intermediary Group IV metal oxide, which, together with the low oxidation potential, will allow formation of a dense and stable Group IV metal oxide without stratification so that the Group IV metal oxide can be oxidised further by a second gaseous species having a higher oxidising potential to create the protective oxide surface layer. The carbon content of the intermediary Group IV metal oxide layer is detectable over the thickness of the intermediary Group IV metal oxide layer, e.g. as a stable concentration over the thickness, but the second oxidation step has surprisingly been found to redistribute, and thereby concentrate, carbon from the intermediary Group IV metal oxide layer to a region of the protective Group IV metal oxide surface layer near the surface of the component. Without being bound by theory, the present inventors believe that redistributing the carbon, e.g. concentrating the carbon near the surface, increases the wear resistance of the protective Group IV metal oxide surface layer compared to the intermediary Group IV metal oxide surface layer. The carbon content may be detected using any appropriate technology. For example, GDOES analysis of the component of the invention will show a carbon content over the thickness of the intermediary Group IV metal oxide and a detectable carbon content in the protective oxide surface layer. The carbon content in the protective oxide surface layer may be detectable over the thickness of the protective oxide surface layer. Other relevant analyses are X-ray photoelectron spectroscopy (XPS), secondary-ion mass spectrometry (SIMS) and Auger electron spectroscopy. Exemplary GDOES analyses are shown in FIG. 10 to FIG. 14. Comparison of FIG. 13 and FIG. 14 shows how the carbon content appears to have migrated towards the surface of the component of the invention. Thus, the carbon content in FIG. 14 decreases from about 100 seconds to 300 seconds of analysis, whereas the carbon content in FIG. 13 appears to increase from the surface to the diffusion zone.

Using substantially pure CO₂ in the first oxidation step will provide the intermediary Group IV metal oxide with sufficient carbon to stabilise the Group IV metal oxide and allow further oxidation of the Group IV metal oxide. However, by increasing the content of CO in the first oxidation step the content of carbon in the intermediary Group IV metal oxide will be increased proportionally. A higher content of carbon in the intermediary Group IV metal oxide can provide a darker colour in the final protective oxide surface layer, and thereby, the component of the invention can be designed for specific uses where a darker colour is relevant.

The method of the invention employs a carbon containing gaseous species and a second gaseous species and the respective reactive conditions may also be referred to as reactive atmospheres. The terms “carbon containing gaseous species” and “second gaseous species” refer to the active compounds with respect to the oxidising reactions, and the reactive atmospheres may also comprise other molecules, e.g. gaseous molecules, that are not active in the oxidising reactions. Gaseous molecules may comprise molecules that are inert, e.g. noble gases, or molecules that may interact with the Group IV metal or alloy but without causing oxidation, e.g. N₂. In the first oxidation step the Group IV metal or alloy is provided with an intermediary Group IV metal oxide. In general, gaseous N₂ will react with Group IV metals and alloys at temperatures of relevance in both the first and second oxidation step. However, in the second oxidation step N₂ cannot immediately reach the Group IV metal but N₂ will include nitrogen into the Group IV metal oxide. The present inventors have now surprisingly found that nitrogen can be used to adjust the colour of the protective oxide surface layer from white to a broken white. In particular, the higher the content of N₂ in the atmosphere, e.g. in the second oxidation step, the more yellow the colour of the protective oxide surface layer. Thereby, the method of the invention is especially suited for making dental implants where the colour of the implant can be closely matched to the colour of the teeth of a patient for whom the implant is designed. In an embodiment of the invention, the second oxidation step and/or the first oxidation step is performed in an atmosphere comprising N₂. When N₂ is present, in particular in the second oxidation step, the protective oxide surface layer will have a content of nitrogen over the thickness of the protective oxide surface layer, which content is generally stable over the thickness. The nitrogen content can be identified using any appropriate analytical method, e.g. GDOES or XPS.

The component of the invention is resistant to wear and, in particular, the treated surface does not experience problems with spallation. In a specific embodiment, the component analysed in a test of wear to be compared to an untreated sample of the same Group IV metal as the component. Thus, the component may have a volumetric loss of up to 5% of the volumetric loss of a component not having the Group IV metal oxide layer of the component of the invention when analysed under identical conditions, e.g. after sliding wear testing under identical conditions. The component is obtainable in the method of the invention and the workpiece before treatment in the method of the invention can be compared to the component of the invention. The workpiece should be identical to the component, except that it does not have the Group IV metal oxide layer. In specific embodiments of the invention the volumetric loss for the component is up to 2% or up to 1%, or up to 0.5%, of the volumetric loss for the untreated workpiece when analysed using the same method. Any test for wear is appropriate. An exemplary test for sliding wear is the ASTM G99-17 test (the “Standard test method for wear testing with a pin-on-disc apparatus”). The analysis may also be performed using other approaches. In particular, since the wear of the component of the invention is compared to the wear of an untreated workpiece, any test is appropriate. For example, the component of the invention and the untreated sample can be treated as follows: providing an untreated sample of the same Group IV metal or alloy as the component of the invention, subjecting the component and the untreated sample to a wear resistance analysis using a ball on disc tribology testing in Ringers solution. Specifically, the wear counterpart can be a 6 mm diameter Al₂O₃ ball loaded with a normal force of 5N on a rotating sample disc for a total of 50 meter with a speed of 0.5 cm/s in a Ringers test solution containing 0.12 g/l CaCl₂, 0.105 g/l KCl, 0.05 g/l NaHCO₃ and 2.25 g/l NaCl. The wear can be quantified as the volumetric loss of the two samples and the volumetric loss of the component of the invention can be compared to the untreated sample.

The second oxidation step does not provide a significantly higher hardness than afforded by the first oxidation step, e.g. as shown in FIG. 1. However, the increased carbon content in the protective oxide surface layer near the surface of the component is believed to provide a higher resistance to wear than available from the intermediary oxide surface layer. In an embodiment, the cross-sectional hardness of the Group IV metal oxide layer is at least 700 HV_{0.005,} e.g. at least 800 HV_{0.005.} For example, the Group IV metal or alloy may be a titanium alloy or pure titanium, and the component may have a titanium oxide layer with a cross-sectional hardness of at least 800 HV_{0.005.} In an embodiment, the Group IV metal or alloy is titanium, e.g. of Grade 4 or Grade 2, and the component has a titanium oxide layer with a cross-sectional hardness of at least 800 HV_{0.005}. In general, zirconium oxide will be harder than titanium oxide, and in another embodiment, the Group IV metal or alloy is a zirconium alloy or pure zirconium, and the component has a zirconium oxide layer with a cross-sectional hardness of at least 1000 HV_{0.005.} In an embodiment, the Group IV metal or alloy is zirconium, and the component has a zirconium oxide layer with a cross-sectional hardness of at least 1000 HV_{0.005}.

Both oxidation steps in the method of the invention are performed at temperatures up to 900° C. Thereby, the Group IV metal or alloy, especially titanium, will not deform during the treatment, and a workpiece in its final shape can be treated in the method of the invention.

A temperature of at least 500° C. is required in the first oxidation step, since at temperatures below 500° C. no oxide will form. In general, the higher the temperature, the more reactive the gaseous species. In particular, treatment at temperatures above 900° C. in the first oxidation step will result in excessive oxide formation so that a stable intermediary Group IV metal oxide layer cannot form on the surface of the Group IV metal or alloy. The first oxidation step is preferably performed at a temperature in the range of 700° C. to 800° C. Temperatures up to 800° C. provide a more stable intermediary Group IV metal oxide than temperatures above 800° C. An especially preferred temperature range for the first oxidation step is 720° C. to 780° C., e.g. about 750° C.

Since the second gaseous species has a higher oxidising potential than the carbon containing gaseous species, the second oxidation step can be performed at a lower temperature than the first oxidation step. For example, the second gaseous species will be reactive towards the intermediary Group IV metal oxide at a temperature of 300° C. In a specific embodiment, the temperature of the second oxidation step is lower than the temperature of the first oxidation step. Thereby, a more cost efficient process is obtained. The second oxidation step is preferably performed at a temperature in the range of 600° C. to 700° C. Temperatures up to 700° C. provide a more stable outer Group IV metal oxide than temperatures above 700° C. An especially preferred temperature range for the second oxidation step is 620° C. to 680° C., e.g. about 650° C. In a particular preferred embodiment, the first oxidation step is performed at a temperature in the range of 700° C. to 800° C., e.g. 720° C. to 780° C., and the second oxidation step is performed at a temperature in the range of 600° C. to 700° C., e.g. 620° C. to 680° C.

The method of the invention allows that a white protective oxide surface layer is formed on the Group IV metal or alloy. When aluminium is present in the Group IV alloy, a stable intermediary oxide of the Group IV metal present in the alloy will be formed, and the intermediary Group IV metal oxide can be converted to the protective oxide surface layer, but the present inventors have surprisingly found that a white surface cannot be obtained in the method of the invention. Without being bound by theory the present inventors believe that aluminium forms stronger oxides than the Group IV metal present in the alloy thereby preventing formation of a white protective oxide surface layer. However, unavoidable impurities of aluminium will not cause problems for the formation of a white oxide of the Group IV metal or alloy. In a preferred embodiment, the Group IV metal or alloy does not comprise aluminium.

Treatment of a Group IV metal or alloy with the carbon containing gaseous species will dissolve oxygen in the Group IV metal or alloy, so that a diffusion zone will form. The oxygen atoms in solid solution in the Group IV metal or alloy will harden the Group IV metal or alloy so that the diffusion zone has a higher hardness than the core of the Group IV metal or alloy. In general, the diffusion zone extends to a depth from the surface of the Group IV metal or alloy where oxygen can be measured and at the interface between the diffusion zone and the intermediary Group IV metal oxide, the Group IV metal or alloy will be saturated with oxygen. When the oxygen content exceeds the saturation level, the intermediary Group IV metal oxide will form. For titanium the diffusion zone has an oxygen content in the range of 1 wt % to 14 wt %. The hardness of a Group IV metal or alloy will correlate directly with the content of oxygen in solid solution. For example, for pure titanium, e.g. grade 2, the core hardness, i.e. at no dissolved oxygen, may be about 200 HV_{0.05} (for pure titanium) and titanium saturated with oxygen may have a hardness of about 1000 HV_{0.05}. In the context of the invention, the limit between the diffusion zone and the core can thus be detected for any Group IV metal or alloy as the depth from the surface where the microhardness is 20% higher than the core hardness.

The component of the invention has a material core having a core hardness, a diffusion zone having oxygen in solid solution in the range of a level providing a hardness of 120% of the hardness of the material core to the saturation level of the Group IV metal or alloy, and a Group IV metal oxide layer at the surface of the component, the diffusion zone being between the Group IV metal oxide layer and the material core. The diffusion zone and the oxide layer can easily be identified in the cross-section of the component by visual observation, e.g. using a microscope, and the diffusion zone can further be defined by the cross-sectional hardness. All three zones may further be defined using GDOES analysis. For example, GDOES can be used to provide a relative comparison of the levels of different elements in the different layers. The treatment with the carbon containing gaseous species in the first oxidation step has been found to introduce carbon in the diffusion zone. Without being bound by theory, the present inventors believe that the carbon content in the diffusion zone provides a stronger integration of the intermediary oxide layer with the core metal and thereby prevents formation of a stratified oxide layer. In the second oxidation step, the carbon in the intermediary oxide layer moves towards the surface of the component, but the carbon in the diffusion zone will not relocate. Thereby, the Group IV metal oxide layer of the component is equally strongly integrated with the core metal, and the Group IV metal oxide layer has, due to the carbon content near the surface, an increased wear resistance compared to the intermediary oxide layer. Thus, the method of the invention provides a component having a wear resistant Group IV metal oxide layer that is strongly integrated with the core metal. The carbon contents of the diffusion zones are illustrated in FIG. 13 and FIG. 14. In a specific embodiment, the diffusion zone contains carbon in solid solution at a level above the carbon content of the Group IV metal oxide layer, e.g. as measured using GDOES. A higher level of carbon in the diffusion zone is believed to provide a more stable integration of the oxide layer on the diffusion zone thereby increasing the stability further.

The relocation of the carbon during the second oxidation step can create a minimum carbon content in the Group IV metal oxide layer of the component, i.e. the protective oxide surface layer. For example, the minimum carbon content is lower than the carbon content near the surface of the protective oxide surface layer. The minimum carbon content is typically detectable at a depth from the surface in the range of 50% to 90% of the thickness of the protective oxide surface layer.

The method of the invention employs a carbon containing gaseous species and a second gaseous species which have a first oxidising potential and a second oxidising potential, respectively. The second oxidising potential is higher than the first oxidising potential. In the context of the invention, the oxidising potentials can be determined by oxidising a reference sample with the two gaseous species under otherwise identical conditions and comparing the thicknesses of the oxide layers after the treatment. The thicker the oxide layer, the higher the oxidising potential. Specifically, the oxidising potential of a gaseous species may be determined in the following steps: providing a reference sample of pure Group IV metal (e.g. pure titanium, such as Grade 1, 2, 3 or 4), oxidising the reference sample at 850° C. using the gaseous species, measuring the thicknesses of the oxide layers, and comparing the thicknesses of the oxide layers obtained in the oxidation. Optionally, a number of reference samples can be analysed at different durations, e.g. 4 to 6 reference samples are analysed over at period in the range of 10 minutes to 1000 minutes.

In general, commonly used gaseous species may be listed according to their oxidising potentials as follows: CO₂<O₂<N₂O. The oxidising potentials of CO₂, ambient air, O₂ and N₂O are illustrated in FIG. 2. H₂O is also contemplated as an oxidising gaseous species. However, it is preferred that the reactive atmospheres in the first oxidation step and the second oxidation step, in particular the first oxidation step, do not comprise hydrogen containing species, since hydrogen may reduce the stability of the intermediary Group IV metal oxide. Furthermore, the presence of interstitial hydrogen in the Group IV metal or alloy will lead to embrittlement of the Group IV metal or alloy, which is undesirable. Thus, it is preferred that no hydrogen containing gaseous species are employed.

An oxidising potential of a gaseous species may be decreased by decreasing the partial pressure of the gaseous species, and an oxidising potential of a gaseous species may be increased by increasing the partial pressure of the gaseous species. The partial pressure may be modified by increasing or decreasing the total pressure in the reaction. However, it is preferred to perform the oxidising reactions at ambient pressure. Operation at ambient pressure simplifies the process compared to operation at amended pressures. Alternatively, the partial pressure of a gaseous species may be lowered by including further gaseous molecules, e.g. inert gasses. A preferred inert gas is argon, although any noble gas may be used.

The pressures employed in the first and the second oxidising steps may be chosen freely. For example, the carbon containing gaseous species in the first oxidising step may be used without further gaseous species present, and the pressure, i.e. the pressure of the carbon containing gaseous species, may be at ambient pressure. However, it is also possible to decrease the pressure of the carbon containing gaseous species, e.g. the carbon containing gaseous species may be included in an atmosphere at ambient pressure with the partial pressure of the carbon containing gaseous species being reduced due to the presence of further gaseous species, e.g. inert gasses. It is also possible for the carbon containing gaseous species to be present at a reduced total pressure without further gaseous species being present. The total pressure may also be increased regardless of the presence of further gaseous species. For example, the carbon containing gaseous species may be at a pressure in the range of 0.1 bar to 5 bar. Likewise, the second gaseous species may be at a pressure in the range of 0.1 bar to 5 bar, e.g. at ambient pressure, with the pressure being controlled by including a further gaseous species, e.g. an inert gas, such as argon, and/or by modifying the total pressure.

In a specific embodiment, the carbon containing gaseous species is CO₂. In another embodiment, the carbon containing gaseous species is a mixture of CO₂ and CO. Specifically, certain gaseous species can take part in reactions with other species in the gaseous phase, e.g. CO₂ and CO can take part in a reaction, which reaction can modify the oxidising potential by modifying the amount of the gaseous species. For example, the carbon containing gaseous species may be a mixture of CO₂ and CO, and at relevant temperatures CO₂ and CO will take part in Reaction 1 and Reaction 2 identified below.

CO(g)+½O₂(g)=CO₂(g) Reaction 1

2CO(g)=CO₂(g)+C Reaction 2

In general, CO is considered to be a carbon providing molecule that does not have an oxidising potential, whereas CO₂ has an oxidising potential. Thus, an oxidising atmosphere of only CO₂ can be considered to provide pure oxidation, whereas 100% CO can be considered to correspond to an infinitely high carbon activity. For example, FIG. 3 shows how an oxide layer could not be formed by treating pure titanium with CO at 840° C. The partial pressure of O₂ (pO₂) and the activity of carbon (a_{c}) can be determined from Equation 1 and Equation 2, so that partial pressure of O₂ is:

$${pO}_{2} = {( \frac{{p CO}_{2}}{p CO} )}^{2} exp ( \frac{2 Δ {G}_{1}}{R T} ) \qquad Equation 1$$

and the activity of carbon is:

$${a}_{C} = {( \frac{p CO}{{p CO}_{2}} )}^{2} exp ( \frac{- Δ {G}_{2}}{R T} ) \qquad Equation 2$$

where ΔG₁=−282.200+86.7 T (J), and ΔG₂=−170.550+174.3 T (J).

By using a mixture of CO₂ and CO, the first oxidising potential can be lowered, and it is therefore possible to tailor the oxidising potential of the gaseous species, e.g. as a mixture of CO₂ and CO, and also control the contents O and C in the Group IV metal oxide, e.g. the titanium oxide. When the first reactive gaseous species contains CO, the carbon activity of CO will dissolve carbon atoms interstitially into the Group IV metal or alloy. Carbon will generally increase the microhardness of an oxygen containing diffusion zone compared to a diffusion zone comprising oxygen but no carbon. Moreover, without being bound by theory the present inventors believe that carbon in the intermediary Group IV metal oxide will further stabilise the intermediary Group IV metal oxide thereby yielding an improved process compared to using only CO₂ as the carbon containing gaseous species. In a specific embodiment, the carbon containing gaseous species is therefore a mixture of CO₂ and CO with 40% to 90% CO₂ compared to the total of CO₂ and CO. In another embodiment, the carbon containing gaseous species is a mixture of CO₂ and CO with 40% to 60% CO₂ compared to the total of CO₂ and CO.

Formation of the diffusion zone will start immediately when the first oxidation step is commenced, and after a reactive duration the oxygen atoms will form the intermediary Group IV metal oxide so that the diffusion zone will be between the core of the Group IV metal or alloy and the intermediary Group IV metal oxide. The reactive duration of the first oxidising step may also be referred to as the first reactive duration. In general, a reactive duration of at least 0.2 hours, e.g. at least 1 hour, is required to form a sufficient layer of the intermediary Group IV metal oxide. The thickness of both the diffusion zone and also the intermediary Group IV metal oxide will depend on the reactive duration. The second oxidation step will generally not increase the thickness of the Group IV metal oxide layer. The dissolution of elements into a Group IV metal is considered to be parabolic so that a doubling of the dissolution depth requires a four times longer reactive duration. However, the reactive duration of the first oxidation step should not be longer than 30 hours. It is preferred that the hardened surface layer, e.g. the Group IV metal oxide layer, has a thickness in the range of 20 μm to 100 μm; for example, in an embodiment of the invention the reactive duration of the first oxidation step is in the range of 1 hour to 24 hours. In a preferred embodiment, the reactive duration of the first oxidation step is in the range of 12 hours to 24 hours, e.g. 14 hours to 18 hours.

The second oxidation step will provide an outer Group IV metal oxide layer, and the second oxidation step may be performed over a reactive duration, i.e. a second reactive duration. In general, a second reactive duration of 0.5 hour will provide a sufficient outer Group IV metal oxide layer. It is preferred that the second reactive duration is in the range of 1 hour to 24 hours, e.g. 12 hours to 20 hours.

In a specific embodiment, the carbon containing gaseous species is CO₂ or a mixture of CO₂ and CO, e.g. at a ratio of CO₂ to CO and CO₂ in the range of 0.4 to 0.9, the first oxidation step temperature is in the range of 700° C. to 800° C., e.g. 720° C. to 780° C., and the first reactive duration is in the range of 12 hours to 24 hours, the second gaseous species is N₂O or O₂, and the second oxidation step temperature is in the range of 600° C. to 700° C., e.g. 620° C. to 680° C., and the second reactive duration is in the range of 12 hours to 24 hours. In this embodiment, the Group IV metal or alloy may be titanium, e.g. grade 1 to 4, zirconium, e.g. Zr702, or a titanium/niobium alloy, e.g. Ti13Nb13Zr. Certain alloying elements can increase or decrease the alpha-to-beta transition (T_{β}) temperature, and specific alloying elements may be included in the Group IV alloy.

In a specific embodiment, the carbon containing gaseous species is CO₂ or a mixture of CO₂ and CO, e.g. at a ratio of CO₂ to CO and CO₂ in the range of 0.4 to 0.6, the first oxidation step temperature in the range of 700° C. to 800° C., e.g. 720° C. to 780° C., and the first reactive duration is in the range of 12 hours to 24 hours, and the second gaseous species is N20 in N2 at a ratio of 20% to 40% N20 to the total of N₂O in N₂, the second oxidation step temperature is in the range of 600° C. to 700° C., e.g. 620° C. to 680° C., and the second reactive duration is in the range of 12 hours to 24 hours. In this embodiment, the Group IV metal or alloy may be titanium, e.g. grade 1 to 4, zirconium, e.g. Zr702, or a titanium/niobium alloy, e.g. Ti13Nb13Zr.

The component is obtainable in the method of the invention, and in particular all advantages observed for components provided in the method of the invention are also relevant for the component of the invention, and the features and the corresponding advantages discussed above for the method aspect are also relevant for the component.

In general, all variations and features for any aspect and embodiment of the invention may be combined freely. The features described above for the method are thus equally relevant for the component of the invention.

### BRIEF DESCRIPTION OF THE FIGURES

In the following the invention will be explained in greater detail with the aid of an example and with reference to the schematic drawings, in which

FIG. 1 shows the hardness of titanium oxides as intermediary and surface oxide layers;

FIG. 2 shows the oxidising potential of a range of gaseous species;

FIG. 3 shows treatment of titanium in CO;

FIG. 4 shows titanium samples before and after treatment;

FIG. 5 shows a cross-section of titanium after the first oxidation step;

FIG. 6 shows a cross-section of titanium after the second oxidation step; step

FIG. 7 shows a cross-section of titanium after the first oxidation step;

FIG. 8 shows a cross-section of titanium after the second oxidation step;

FIG. 9 shows a hardness analysis in the cross-section of titanium hardened according to the invention;

FIG. 10 shows Glow Discharge Optical Emission Spectroscopy (GDOES) of untreated titanium;

FIG. 11 shows GDOES of titanium treated in the first oxidation step of the invention;

FIG. 12 shows GDOES of titanium hardened according to the invention;

FIG. 13 shows an enlarged section of the GDOES of FIG. 11;

FIG. 14 shows an enlarged section of the GDOES of FIG. 12;

FIG. 15 shows a spectrophotometric analysis of titanium hardened according to the invention;

FIG. 16 shows a cross-section of a titanium sample oxidised using O₂;

FIG. 17 shows a cross-section of a zirconium component of the invention;

FIG. 18 shows the hardness of a zirconium component of the invention, the zirconium and the intermediary zirconium oxide;

FIG. 19 shows hardness analyses of a workpiece of Ti13Nb13Zr, and the workpiece after the first and second oxidation steps of the invention;

FIG. 20 shows cross-sections of a workpiece of Ti13Nb13Zr, and the workpiece after the first and second oxidation steps of the invention.

Reference to the figures serves to explain the invention and should not be construed as limiting the features to the specific embodiments as depicted.

### DETAILED DESCRIPTION OF THE INVENTION

The present invention relates to a method of producing a protective oxide surface layer on a Group IV metal or alloy component having a Group IV metal oxide layer at the surface of the component.

In the context of the invention “Group IV metal or alloy” is any metal selected from the titanium group of the periodic table of the elements or an alloy comprising at least 50% of metals from the titanium group. Thus, a “titanium alloy” is any alloy containing at least 50% (a/a) titanium, and likewise a “zirconium alloy” is any alloy containing at least 50% (a/a) zirconium. It is contemplated that for the method of the invention and for the component of the invention any alloy containing a sum of titanium and zirconium of at least 50% (a/a) is appropriate. Likewise, the alloy may also comprise hafnium, which is a member of Group IV of the periodic table of the elements so that any alloy having a sum of titanium, zirconium, and hafnium of at least 50% (a/a) is appropriate for the invention.

Alloys of relevance to the invention may contain any other appropriate element, and in the context of the invention an “alloying element” may refer to a metallic element in the alloy, or any constituent in the alloy. Titanium and zirconium alloys are well-known to the skilled person.

Any grade of titanium containing at least about 99% (w/w) titanium is, in the context of the invention, considered to be “pure titanium”, e.g. Grade 1 titanium or Grade 2 titanium; thus, the pure titanium may contain up to about 1% (w/w) trace elements, e.g. oxygen, carbon, nitrogen or other metals, such as iron. In particular, nitrogen and carbon contained in a Group IV metal or alloy in the context of the invention may represent unavoidable impurities. Elements present as “unavoidable impurities” are considered not to provide an effect for a workpiece treated according to the method of the invention or for the component of the invention. Likewise, any grade of zirconium containing at least about 99% (w/w) zirconium is, in the context of the invention, considered to be “pure zirconium”.

When a percentage is stated for a metal or an alloy the percentage is by weight of the weight of material, e.g. denoted wt %, unless otherwise noted. When a percentage is stated for an atmosphere the percentage is by volume, e.g. denoted % (v/v), unless otherwise noted. Likewise, unless otherwise noted a composition of a mixture of gasses may be on an atomic basis and may then be provided as a percentage or in ppm (parts per million).

The method of the invention employs a gaseous species. In particular, the method employs a gaseous species having an oxidising potential. A gaseous species having an oxidising potential may also be referred to as an oxidising species or an oxidising gaseous species. The method may also employ further gaseous molecules, e.g. inert gaseous molecules that are not oxidising. Unless noted otherwise, a “gaseous species” is an oxidising species. When an oxidising species takes part in a reaction with another species, the mixture of the oxidising species and the other species may be referred collectively to as the “gaseous species”, e.g. the carbon containing gaseous species may be a mixture of CO₂ and CO.

In the context of the invention the hardness is generally the HV_{0.05} as measured according to the DIN EN ISO 6507 standard. If not otherwise mentioned the unit “HV” thus refers to this standard. The hardness is preferably recorded for a cross-section, e.g. of a treated Group IV metal or alloy, and it may be noted with respect to the depth from the surface of the measurement. The hardness measurement in the cross-section may also be referred to as “microhardness”, and the hardness measurement at the surface may also be referred to as “macrohardness”.

The microhardness measurement is generally independent of the testing conditions, since the measurement is performed at microscale in the cross-section. Microhardness measurements are typically performed at a load of 25 g, i.e. HV_{0.025,} or 50 g, i.e. HV_{0.05.} In contrast, the macrohardness may be performed from the surface with a much higher load, e.g. 0.50 kg, corresponding to H_{v0.5}, so that the measurement represents an overall value of the hardness of the respective material and whatever surface layers it contains. Microhardness measurements at loads of 25 g or 50 g typically provide the same value, “HV”, but measurement at 25 g is preferred since the measurement requires less space in the cross-section.

When the hardness is recorded at a cross-section the measurement is considered to represent a homogeneous sample with respect to the direction of the pressure applied. In contrast, when the hardness is obtained from measurements at the surface the measurement may represent an average of several different values of hardness, i.e. at different depths. In the context of the invention a hardness measurement recorded in a cross-section at a depth of about 1 μm is considered to provide the actual hardness of the surface of the material. As an effect of the fact that oxygen is dissolved from the surface the content of dissolved oxygen will decrease from the surface towards the core of the Group IV metal or alloy, and likewise, the hardness will be maximal at the surface, e.g. as represented by measuring the hardness in a cross-section at a depth of about 1 μm.

### EXAMPLES

### Comparative Example 1

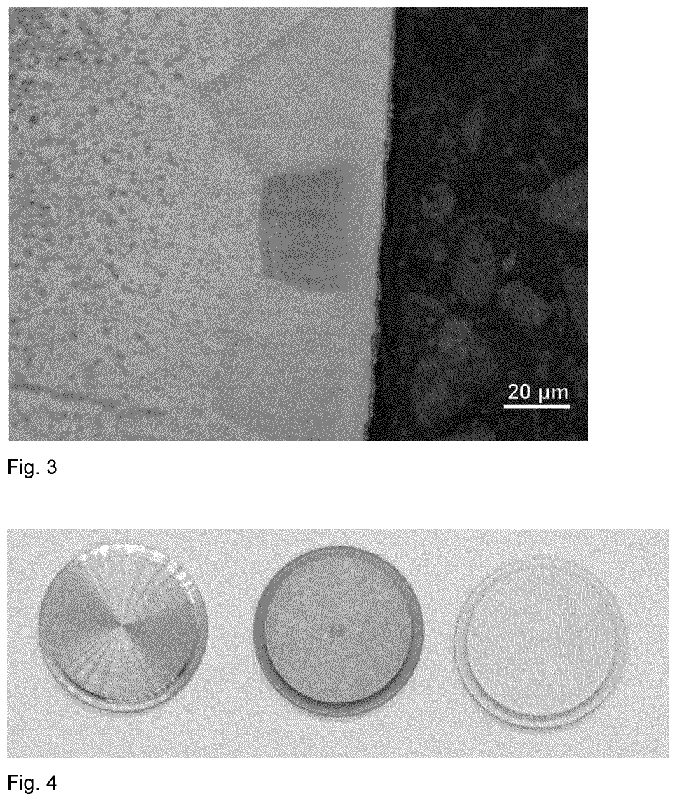

A cylindrical (Ø10 mm) grade 2 titanium samples was treated in a Netzsch 449 Thermal analyzer (furnace). In the experiment, the furnace was evacuated and backfilled with argon gas twice and a continuous gas flow consisting of 10 ml/min Ar and 40 ml/min CO was applied. The sample was heated to 840° C. at a rate of 20° C./min in the same gas mixture and upon reaching the temperature held there for 16 hours. Cooling was carried out at 50° C./min in the flowing process gas. This treatment resulted in formation of a diffusion zone without visible a phase of titanium oxide as is evident from FIG. 3. Thus, without the presence of an oxidising species CO alone cannot provide an oxide layer.

### Example 1

Cylindrical test samples with diameters of 15 mm and thicknesses of 2 mm were treated in oxidising atmospheres. The test samples were CP titanium grade 2. The oxidation was performed at 850° C. in atmospheres of either O₂, Air, N₂O, or CO₂ for between 16 minutes and 900 minutes.

For oxidation in O₂, the test sample was placed in a NETZCH STA449 F3 furnace. The furnace was evacuated and backfilled with O₂. A continuous gas flow of 63 ml/min was used. N₂ was used as a protective gas with a flow of 5 ml/min. The test sample was heated to 850° C. at a rate of 30 K/min. The furnace was kept at 850° C. for 16 minutes after which the furnace was allowed to cool down to room temperature at a rate of 20K/minutes. The experiment was repeated keeping the furnace at 850° C. for 36 minutes, 64 minutes, 400 minutes, and 900 minutes.

For oxidation in Air, the test sample was placed in a Nabertherm LE4/11 R6 furnace. The test sample was then heated to 850° C. at a rate of 30 K/minutes. The furnace was kept at 850° C. for 16 minutes after which the test sample was taken out of the furnace and allowed to cool down to room temperature. The atmosphere in the furnace was air. The experiment was repeated keeping the furnace at 850° C. and taking the samples out after 25 minutes, 36 minutes, 49 minutes, 64 minutes, 400 minutes, and 900 minutes.

For oxidation in N₂O, the test sample was placed in a NETZCH STA449 F3 furnace. The furnace was then evacuated and backfilled with N₂. When reaching ambient pressure, N₂O was added at a continuous gas flow of 15 ml/minutes N₂ was used as a protective gas with a flow of 30 ml/minutes The test sample was heated to 850° C. at a rate of 30 K/minutes The furnace was kept at 850° C. for 16 minutes after which the furnace was allowed to cool down to room temperature at a rate of 20K/minutes The experiment was repeated keeping the furnace at 850° C. for 25 minutes, 36 minutes, 49 minutes, 64 minutes, 225 minutes, 400 minutes, and 900 minutes.

For oxidation in CO₂, the test sample was placed in a NETZCH STA449 C furnace. The furnace was then evacuated and backfilled with CO₂. A continuous gas flow of 50 ml/minutes was used the test sample was heated to 850° C. at a rate of 30 K/minutes The furnace was kept at 850° C. for 16 minutes after which the furnace was allowed to cool down to room temperature at a rate of 20K/minutes. N₂ was used as a protective gas with a flow of 5 ml/minutes The experiment was repeated keeping the furnace at 850° C. for 25 minutes, 36 minutes, 49 minutes, 64 minutes, 400 minutes, and 900 minutes.

The surface showed oxide layers on the surfaces of the CP titanium grade 2 test samples. The test samples were cut in half, embedded and polished. Using light optical microscopy, the thicknesses of the oxide layer were measured and plotted in FIG. 2. The thicknesses obtained for the tested gasses were considered to represent the oxidising potentials of the tested gasses, i.e. the thicker the oxide layer, the higher the oxidising potential. FIG. 2 thus shows a linear relationship in the oxide layer thickness with increasing treatment time and a clear difference in oxidising potential between the four different atmospheres used. Thus, the oxidising potentials of the tested gasses can be ordered as follows: CO₂<air<O₂<N₂O.

### Example 2

Samples of pure titanium (Grade 2) with diameters of 15 mm and thicknesses of 2 mm were treated according to the invention by using CO₂ as the first reactive species at 650° C., 700° C. or 750° C. for 16 hours, and this was followed by oxidation with N₂₀ as the second reactive species at 750° C. for 64 minutes or 400 minutes. Both oxidations were performed at ambient pressure. Specifically, the first oxidation step was performed in a MTI OFT-1200 glass tube furnace. The furnace was evacuated and backfilled with CO₂ at a continuous gas flow of 50 ml/min before heating the test sample to the tested temperatures at a rate of 12 K/min. The second oxidation step was performed in a NETZCH STA449 F3 furnace. After placing the samples in the furnace, the furnace was evacuated and backfilled with N₂. A continuous gas flow of 15 ml/min N₂O was used. N₂ was used as a protective gas with a flow of 30 ml/min. The furnace was kept at 750° C., 700° C., or 650° C. for 400 min or 64 min after which the furnace was allowed to cool down to room temperature at 20 K/min.

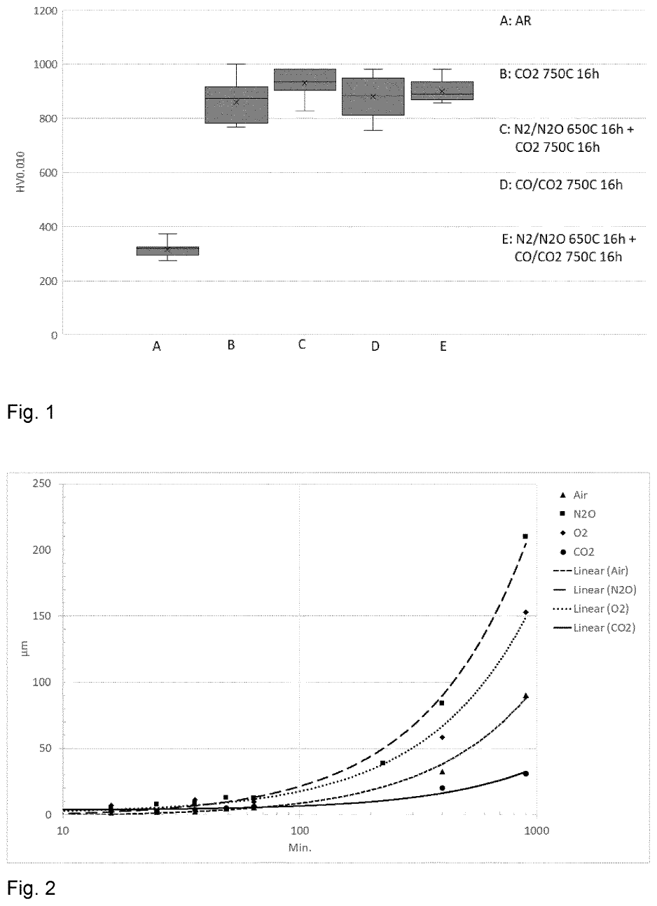

Following the two oxidations, the samples were analysed for surface hardness (HV_{0.010}). For comparison, a sample not exposed to the second oxidation step was also analysed (this sample was treated in CO₂ at 750° C.) for surface hardness. The hardness measurements are shown in FIG. 1, where the “boxes” show the measurements with standard deviations, and the “bars” show the confidence intervals (α=0.05). Thus, the intermediary titanium oxide and the protective oxide surface layers were considerably harder than the core hardness (of about 300 HV). The second oxidation step treatment did not provide a significantly higher hardness to the outer titanium oxide layer on the surface than the first oxidising step. The intermediary titanium oxide was dense and robust, but the protective oxide surface layer had a higher resistance to wear than the intermediary titanium oxide layer.

### Example 3

Samples of pure titanium were hardened according to the invention or in a treatment using only N₂O. Specifically, in the first oxidation step the samples were placed in a MTI OFT-1200 glass tube furnace before evacuating and backfilling with the appropriate gas. A continuous gas flow of 200 ml/min was used, and the test samples were heated at a rate of 12 K/min. After this first step, the furnace was allowed to cool down to room temperature unaided. The treatment in the second oxidation step was done separately from the first oxidation step although the same furnace was used. After placing the intermediary workpiece in the furnace, the furnace was evacuated and backfilled with the appropriate gas at continuous gas flow of 300 ml/min, and the intermediary workpiece was heated at a rate of 12 K/min. After the second oxidation step, the component the furnace was allowed to cool down to room temperature unaided.

Thus, a sample was treated in CO₂ at ambient pressure and 780° C. for 16 hours followed by treatment in N₂O at 680° C. for 3 hours, also at ambient pressure. Treatment in N₂O alone without hardening with CO₂ was performed at 880° C. for 16 hours. The treated samples and an untreated titanium sample were analysed for wear resistance by performing a ball on disc tribology testing in Ringers solution. Specifically, the wear counterpart was a 6 mm diameter Al₂O₃ ball loaded with a normal force of 5N on the rotating sample disc for total 50 meter with a speed of 0.5 cm/s. The test solution was Ringers solution containing 0.12 g/l CaCl₂, 0.105 g/l KCl, 0.05 g/l NaHCO₃ and 2.25 g/l NaCl. The results are shown in Table 1.

TABLE 1
Wear resistance test and comparison

| Sample | Volume loss [mm³] |
|---|---|
| Untreated reference | 0.3170 |
| N₂O 880° C. 16 h | 1.4290 |
| CO₂ 780° C. 16 h + N₂O 680° C. 3 h | 0.0011 |

As seen from Table 1, hardening according to the invention significantly improved the wear resistance compared to the untreated sample. In contrast, treatment in N₂O provided a worse performance than the untreated sample.

Specifically, the treatment in N₂O at 880° C. resulted in a stratified titanium oxide layer that was highly susceptible to spallation. In contrast, the component of the invention had a volumetric loss of 0.35% of the volumetric loss of the untreated workpiece.

### Example 4

Samples of commercially pure (CP) titanium (Grades 2 and 4) with diameters of 15 mm and thicknesses of 2 mm were hardened according to the invention. In the first oxidation step, the samples were placed in a MTI OFT-1200 glass tube furnace. The furnace was evacuated and backfilled with CO₂. A continuous gas flow of 200 ml/min was used. The test sample was heated to 750° C. at a rate of 12 K/min. The furnace was kept at 750° C. for 16 hours after which the furnace was allowed to cool down to room temperature unaided.

In the second oxidation step, test sample was again placed in the MTI OFT-1200 glass tube furnace. The furnace was evacuated and backfilled with 30% N₂O and 70% N₂. A continuous gas flow of 300 ml/min total was used. The test sample was heated to 650° C. at a rate of 12 K/m in. The furnace was kept at 650° C. for 16 hours after which the furnace was allowed to cool down to room temperature unaided.

Photographs of the untreated sample, the intermediary sample and the final sample (Grade 4 titanium) are shown in FIG. 4. The intermediary test sample had a dense robust grey oxide layer, and the final component had a surface showing a white, dense, hard, and robust oxide layer.

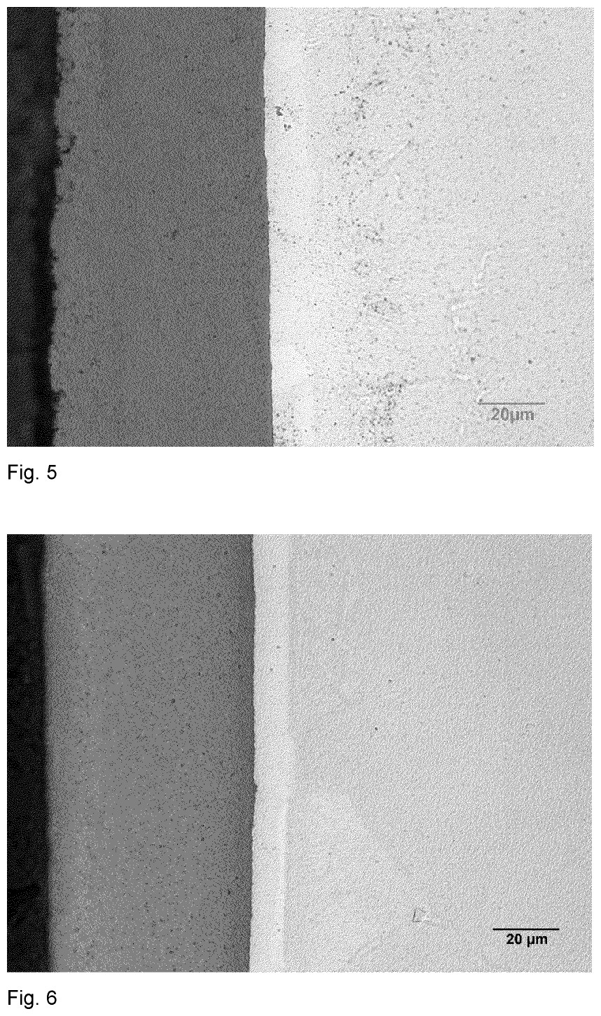

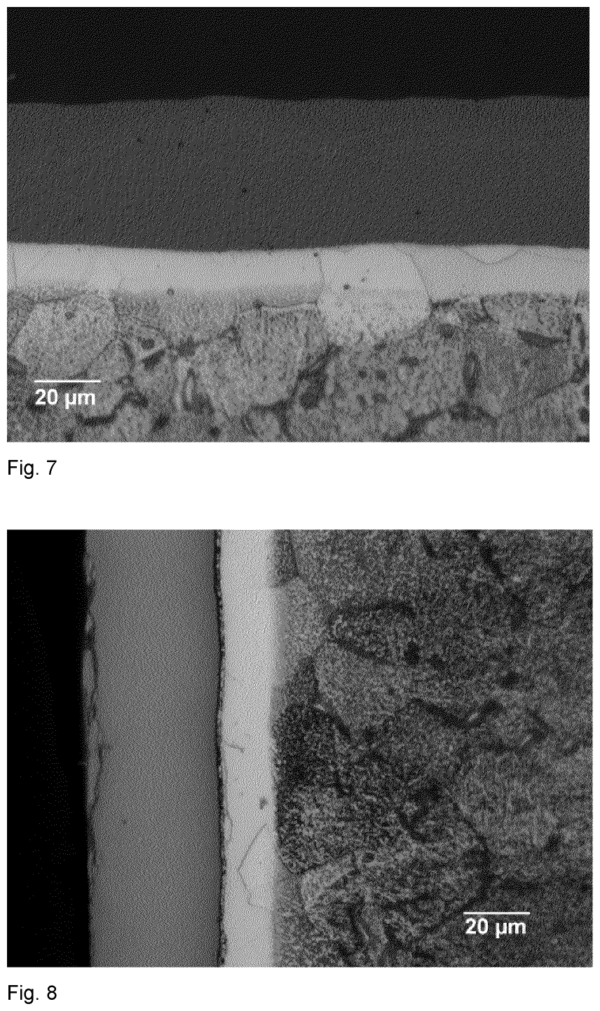

For both Grade 2 and Grade 4 titanium the cross-sections of the samples were analysed after the first and the second oxidation steps. The results are depicted FIG. 5 to FIG. 8, where FIG. 5 shows the Grade 4 titanium sample after the first oxidation step, FIG. 6 shows the Grade 4 titanium sample after the second oxidation step, FIG. 7 shows the Grade 2 titanium sample after the first oxidation step, and FIG. 8 shows the Grade 2 titanium sample after the second oxidation step. It is seen that for both Grades the intermediary oxide layer and the final oxide layer, i.e. the protective oxide surface layer, have the same approximate thickness, whereas the oxide layers are thicker for Grade 4 titanium compared to Grade 2 titanium. Without being bound by theory, the present inventors believe that the higher thickness, about 60 μm, for the Grade 4 titanium compared to the thickness of about 20 μm for Grade 2 titanium can be attributed to a higher content of iron in Grade 4 titanium compared to Grade 2 titanium: Specifically, the inventors believe that iron increases the speed of the reaction so that a thicker oxide layer will form on

Grade 4 titanium compared to Grade 2 titanium. However, for both Grades of titanium the intermediary oxide layers were dense and allowed formation of sufficiently thick protective oxide surface layers.

### Example 5

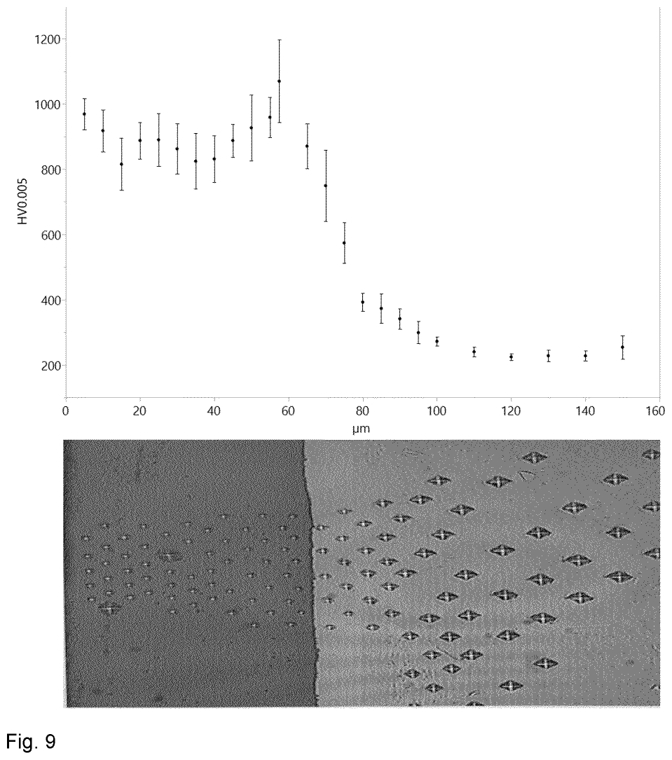

The Grade 4 sample of Example 4 was selected for further analysis, and the cross-section was thus analysed for hardness. Hardness measurements were performed using a 5 g load, and the hardness measurements are shown in FIG. 9, which also shows the actual cross-section after analysis. The protective oxide surface layer had a hardness >800 HV_{0.005}, and at a depth of about 60 μm the interface between the oxide layer and the diffusion zone is visible. The hardness is seen to drop from a level near the interface suggesting titanium saturated with oxygen, i.e. >800 HV_{0.005} to a level of 120% of the hardness of the core metal at a depth of about 100 μm.

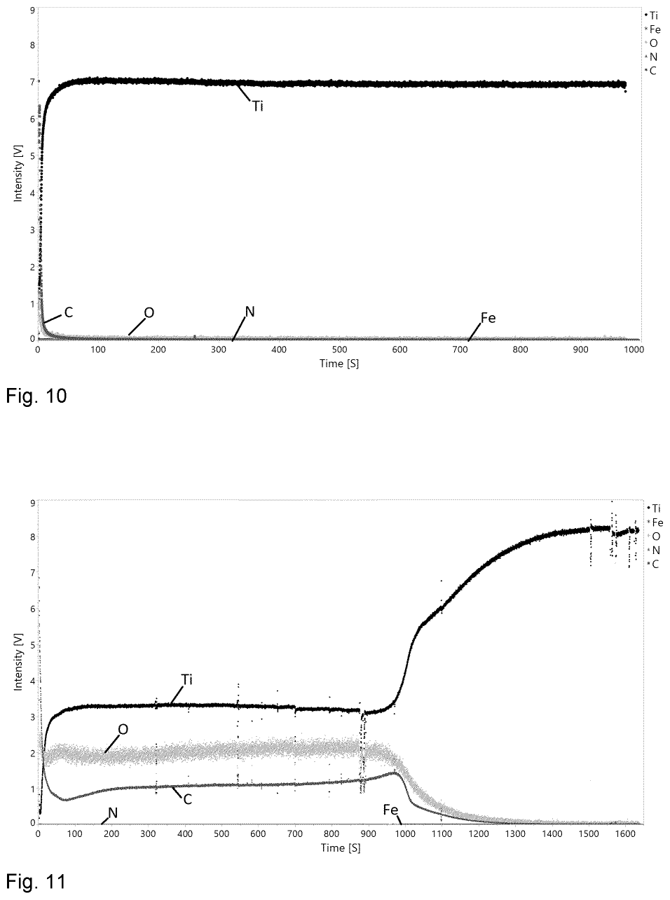

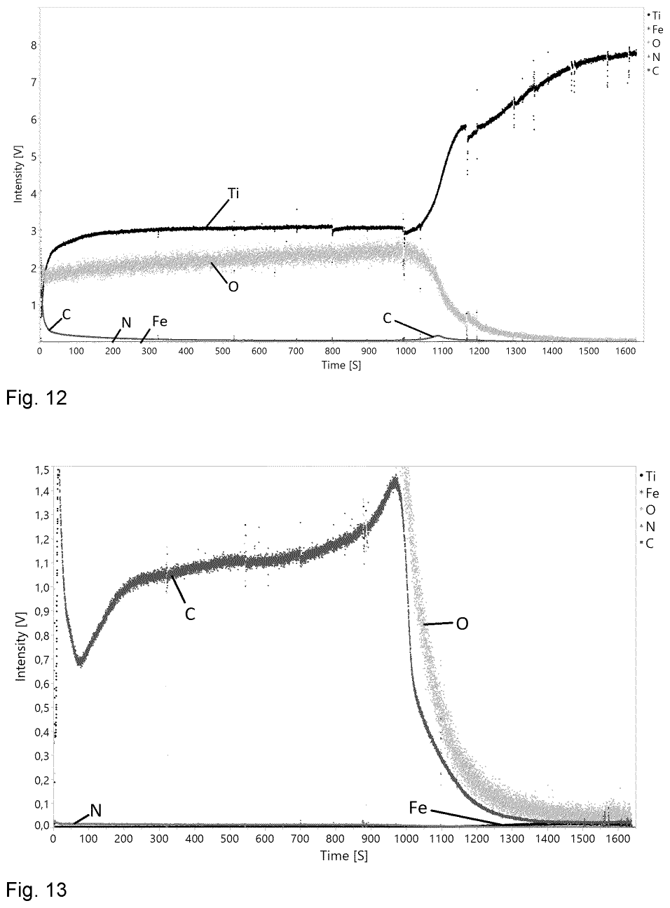

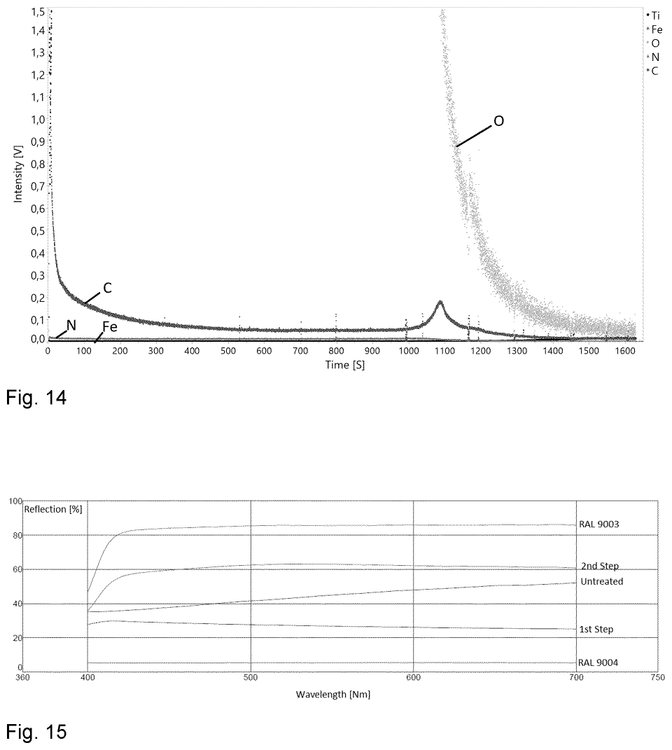

The untreated Grade 4 titanium sample, the Grade 4 titanium sample with the intermediary oxide layer, and the hardened Grade 4 titanium sample were also exposed to Glow Discharge Optical Emission Spectroscopy (GDOES) analysis. The GDOES analyses the content of specified elements shown as an intensity (in the unit V) over time (in second). Thus, the intensity reflects the relative amount of the element and the time reflects the depth from the surface. By analysing the sample for a sufficient time to reflect the composition of all three layers, the GDOES analysis appropriately provides a comparison of the compositions of the protective oxide surface layer (in the left side of the plot) with the diffusion layer (middle section of the plot) and the core of the metal (right section of the plot). Thus, FIG. 10 shows a GDOES analysis of untreated titanium, FIG. 11 shows a GDOES analysis of titanium after the first oxidation, and FIG. 12 shows a GDOES analysis of titanium after the second oxidation. FIG. 13 and FIG. 14 show enlarged sections of FIG. 11 and FIG. 12, respectively. FIG. 10 initially, i.e. in the first 50 seconds, shows apparent sharp decreases in contents of non-metallic elements, which are considered to represent inevitable contents of these elements in titanium. The initial sharp decreases are also observed after the first and second oxidation steps (FIG. 11, FIG. 12, FIG. 13 and FIG. 14). FIG. 11 and FIG. 12 show a content of carbon over the thickness of the intermediary titanium oxide layer, and FIG. 13 and FIG. 14 shows how the carbon content appears to have migrated towards the surface of the component of the invention. The drop in oxygen signal and the increase in titanium signal is considered to represent the diffusion zone, and the signals after about 1300 seconds are considered to represent the metal core. Enlarged sections of the plots in FIG. 11 and FIG. 12 are shown in FIG. 13 and FIG. 14, respectively, where FIG. 11 and FIG. 12 show 8 intensity units on the Y-axis and FIG. 14 show 1.5 intensity units on the Y-axis. FIG. 12 shows that the protective oxide surface layer has a composition of mainly titanium and oxygen, and at the interface between the protective oxide surface layer and the diffusion zone the oxygen content drops towards the metal core. The enlarged section in FIG. 14 highlights that the protective oxide surface layer has a detectable content of carbon over the thickness of the protective oxide surface layer, although the carbon content is higher near the surface of the component. The titanium sample was treated with CO₂ in the first oxidation step to that the carbon content seen for the protective oxide surface layer was also present in the intermediary oxide layer. Without being bound by theory the present inventors believe that this detectable content of carbon stabilises the intermediary oxide layer and allows that the intermediary oxide layer is oxidised further in the second oxidation step to provide a protective oxide surface layer that is as stable as the intermediary oxide layer but harder than the intermediary oxide layer. The increased hardness of the protective oxide surface layer compared to the intermediary oxide layer provides a component with wear resistance beyond what has been available in methods of the prior art.

FIG. 14 further shows that the diffusion zone, especially near the interface with the protective oxide surface layer has an increased amount of carbon compared to both the content in the metal core and also the protective oxide surface layer. Without being bound by theory, the present inventors believe that this carbon, i.e. present in solid solution in the diffusion zone, further stabilises the protective oxide surface layer on the surface of the hardened component thereby contributing to the stability of the protective oxide surface layer on the component of the invention.

The second oxidation step was performed in an atmosphere of N₂O in N₂. The presence of N₂ in the second oxidation step has provided the protective oxide surface layer with a stable content of nitrogen, as shown by the GDOES analysis in FIG. 14. The level of the nitrogen content can be controlled by controlling the amount of N₂ in the second oxidation step, but regardless of the N₂ in the second oxidation step, the amount of nitrogen in the protective oxide surface layer will be stable over its thickness. The present inventors have now surprisingly found that nitrogen in the protective oxide surface layer will change its colour from white towards a more yellow tinge, and furthermore that modification of the content of N₂ in the second oxidation step can be used to adjust the colour of the protective oxide surface layer from white to a broken white; the higher the content of N₂ in the second oxidation step, the more yellow the colour of the protective oxide surface layer.

### Example 6

The surface of the Grade 4 sample of Example 4 was analysed spectrophotometrically and compared to a standard white, RAL9003 (“signal white”), and black, RAL9004 (“signal black”) together with an untreated sample and a sample after the first oxidation step. The component of the invention is seen to have a white and reflective surface as indicated by the high reflectance (>60%) and the little variation in reflectance over the visible range of wavelengths.

### Example 7

A sample of Ti6Al4V alloy with diameters of 15 mm and thicknesses of 2 mm was treated according to the invention in two separate oxidation steps. In the first oxidation step, the sample was placed in a MTI OFT-1200 glass tube furnace. The furnace was evacuated and backfilled with CO₂. A continuous gas flow of 200 ml/min was used. The test sample was then heated to 750° C. at a rate of 12 K/m in. The furnace was kept at 750° C. for 16 hours after which the furnace was allowed to cool down to room temperature unaided. The intermediate test sample at this point had a dense robust grey oxide layer.

In the second oxidation step, test sample was again placed in the MTI OFT-1200 glass tube furnace. The furnace was evacuated and backfilled with 30% N₂O and 70% N₂. A continuous gas flow of 300 ml/min total was used. The test sample was heated to 750° C. at a rate of 12 K/m in. The furnace was kept at 650° C. for 16 hours after which the furnace was allowed to cool down to room temperature unaided.

The surface of the sample after the final oxidation step displayed a grey dense, hard, and robust oxide layer. The oxide layer has a thickness of 10 to 15 μm. The oxide does not turn white when applying this thermochemical treatment due to the presence of N₂.

### Example 8

In a variant of the invention, ambient air was used as the second gaseous species for the treatment of CP titanium of grade 4. A samples with a diameter of 15 mm and a thickness of 2 mm was placed in a MTI OFT-1200 glass tube furnace. The furnace was evacuated and backfilled with CO₂. A continuous gas flow of 200 ml/min was used. The test sample was then heated to 750° C. at a rate of 12 K/m in. The furnace was kept at 750° C. for 16 hours after which the furnace was allowed to cool down to room temperature unaided. The test sample has at this point a dense robust grey oxide layer.

In the second oxidation step, test sample was placed in a Nabertherm N 7/H furnace. The test sample was heated to 650° C. at a rate of 12 K/min. The furnace was then kept at 650° C. for 16 hours after which the furnace was allowed to cool down to room temperature unaided. The atmosphere in the furnace was ambient air at ambient pressure. No circulation of the air was used.

The thus provided component had a white, dense, hard, and robust oxide layer on the surface, which was similar in appearance to the surface seen using N₂O in second step.

### Example 9

The effect of using O₂ as the first oxidising species in the treatment of CP titanium of grade 2 was analysed. A sample with a diameter of 15 mm and a thickness of 2 mm was placed in a NETZCH STA449 F3 furnace. The furnace was evacuated and backfilled with O₂. A continuous gas flow of O₂ of 63 ml/min was used together with a flow of N₂ of 5 ml/min. N₂ was contemplated as a protective gas considering the strong oxidising potential of O₂. The test sample was heated to 850° C. at a rate of 30 K/min. The furnace was kept at 850° C. for 400 min after which the furnace was allowed to cool down to room temperature at a rate of 20 K/min.

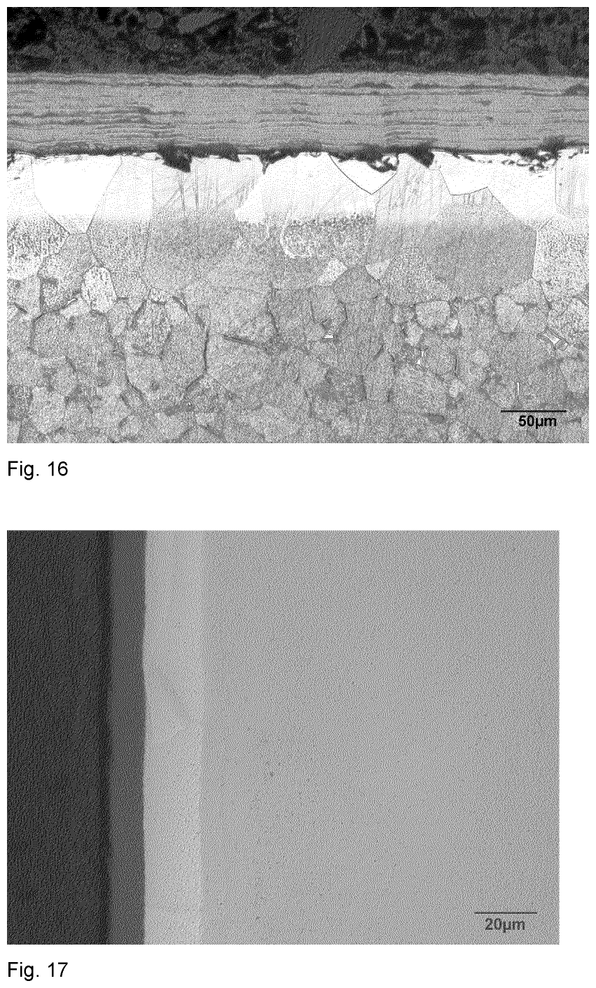

The treatment with O₂ provided the surface with a white oxide layer with a thickness of 50 to 55 μm as seen on the light optical images. The white oxide layer had a hardness of 350-700 HV_{0.025}, but the oxide layer showed a stratified structure indicating a low resistance to spallation. The cross-section of the treated sample is shown in FIG. 16. The oxide layer was thus observed to easily detach from the surface when handled.

### Example 10

The effect of using ambient air as the first oxidising species in the treatment of CP titanium of grade 2 was analysed. The procedure of Example 9 was repeated but with ambient air in place of the mixture of O₂ with N₂. Since ambient air was used, no flow through the furnace was employed.

This treatment provided a white oxide layer having a thickness of 25 to 30 μm and an apparent hardness of 700 to 1000 HV_{0.025}. However, as for Example 9, the oxide layer showed a stratified structure indicating a low resistance to spallation. The oxide layer was thus observed to easily detach from the surface when handled.

### Example 11

The effect of using N₂O as the first oxidising species in the treatment of CP titanium of grade 2 was analysed. A sample with a diameter of 15 mm and a thickness of 2 mm was placed in a NETZCH STA449 F3 furnace. The furnace was evacuated and backfilled with N₂O. A continuous gas flow of 15 ml/min was used with a flow of N₂ of 30 ml/min. The test sample was heated to 750° C. at a rate of 30 K/min, and the furnace was kept at 750° C. for 400 minutes after which the furnace was allowed to cool down to room temperature at a rate of 20 K/min.

The procedure was repeated for a new, untreated sample with a treatment temperature of 1000° C. and a duration of 64 minutes.

Both treatments provided white oxide layers having a thickness of 15 to 20 μm and 60 to 65 μm, respectively, and apparent hardnesses of 700 to 1000 HV_{0.025}. However, as for Examples 9 and 10, the oxide layers showed a stratified structure indicating a low resistance to spallation. The oxide layer was thus observed to easily detach from the surface when handled.

### Example 12

A square test sample of Zr702 with a side length of 15 mm and a thickness of 1.5 mm was treated in two separate oxidation steps according to the invention. In the first oxidation step, the sample was placed in a MTI OFT-1200 glass tube furnace, which was evacuated and backfilled with CO₂. A continuous gas flow of 200 ml/min was used. The test sample was then heated to 750° C. at a rate of 12 K/m in. The furnace was kept at 750° C. for 16 hours after which the furnace was allowed to cool down to room temperature unaided. The intermediary test sample at this point had a dense robust grey oxide layer.

In the second oxidation step, the sample was again placed in the same furnace that was evacuated and backfilled with 30% N₂O and 70% N₂. A continuous total gas flow of 300 ml/min was used. The test sample was heated to 750° C. at a rate of 12 K/m in. The furnace was kept at 650° C. for 16 hours after which the furnace was allowed to cool down to room temperature unaided.

The provided component had a grey dense, hard, and robust oxide layer with a thickness of 8 to 12 μm. The cross-section of the zirconium component is shown in FIG. 17.

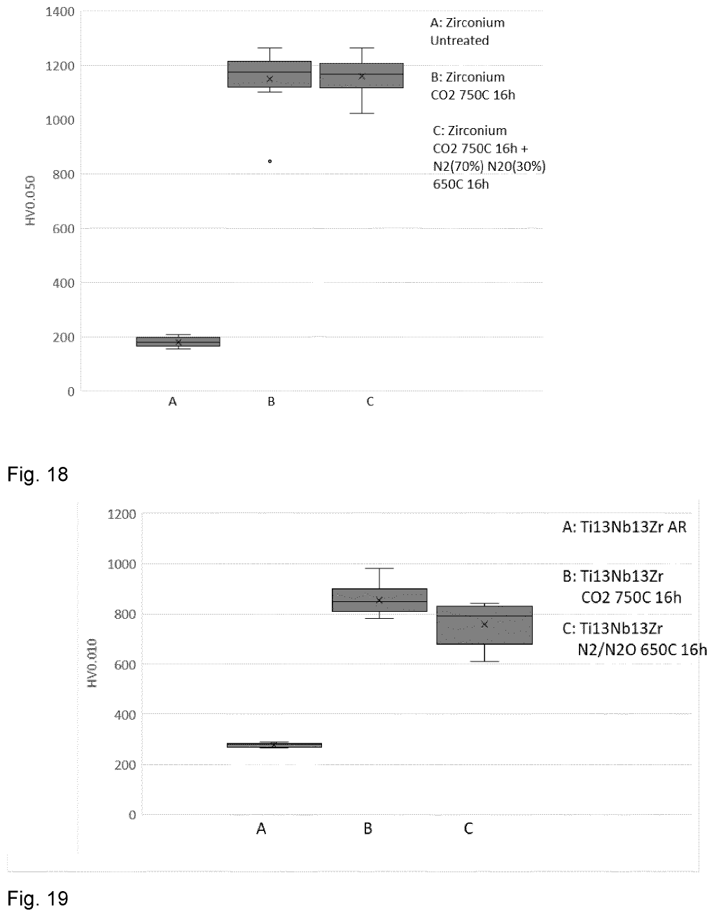

The hardened component, the workpiece with the intermediary zirconium oxide and the workpiece prior to treating in the method of the invention were analysed for surface hardness, and the results are depicted in FIG. 18. The untreated zirconium had a hardness of about 200 HV_{0.05}, whereas the treated zirconium component had a surface hardness of about 1150 HV_{0.05}.

### Example 13

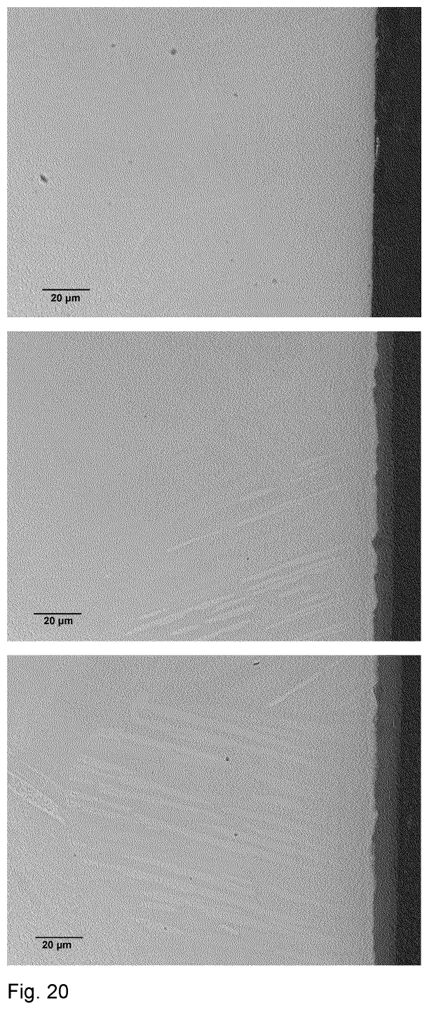

A sample of Ti13Nb13Zr was treated as outlined in Example 4. Cross-sections of the workpiece before treatment, the workpiece after the first oxidation step and the final component are shown in FIG. 20 in the top panel, the middle panel and the bottom panel, respectively. Thus, the workpiece did not have a visible oxide layer, whereas the first oxidation step provided a dense intermediary oxide layer. The hardness values found in FIG. 19 show that the hardness increased upon oxidation. The component had a more stable oxide layer than the workpiece after the first oxidising step.

1. A Group IV metal or alloy component having a protective oxide surface layer, the Group IV metal or alloy component comprising a material core having a core hardness, a diffusion zone having oxygen in solid solution in the range of a level providing a hardness of 120% of the hardness of the material core to the saturation level of the Group IV metal or alloy, and a Group IV metal oxide layer at the surface of the component, the diffusion zone being between the Group IV metal oxide layer and the material core,
wherein the Group IV metal oxide layer has a thickness in the range of 5 μm to 100 μm, a carbon content and a cross-sectional hardness of at least 650 HV_{0.005}.

2. The Group IV metal or alloy component according to claim 1, wherein the diffusion zone contains carbon in solid solution at a level above a minimum carbon content of the Group IV metal oxide layer.

3. The Group IV metal or alloy component according to claim 1, wherein the Group IV metal oxide layer contains nitrogen.

4. The Group IV metal or alloy component according to claim 1, wherein the component has a volumetric loss of up to 5% of the volumetric loss of a component not having the Group IV metal oxide layer when analysed after sliding wear testing under identical conditions.

5. The Group IV metal or alloy component according to claim 1, wherein the Group IV metal or alloy is selected from the list consisting of titanium, a titanium alloy, zirconium, and a zirconium alloy.

6. The Group IV metal or alloy component according to claim 1, wherein the Group IV metal or alloy does not comprise aluminium.

7. The Group IV metal or alloy component according to claim 1, wherein the component has a core of another material.

8. The Group IV metal or alloy according to claim 1, wherein the component is a titanium alloy or pure titanium and has a titanium oxide layer with a cross-sectional hardness of at least 800 HV_{0.005}.

9. The Group IV metal or alloy according to claim 1, wherein the Group IV metal or alloy is a titanium/niobium alloy.

10. The Group IV metal or alloy according to claim 1, wherein the Group IV metal or alloy is a zirconium alloy or pure zirconium, and the component has a zirconium oxide layer with a cross-sectional hardness of at least 1000 HV_{0.005}.

11. A method of producing a protective oxide surface layer on a Group IV metal or alloy, the method comprising the steps of:
providing a workpiece of a Group IV metal or alloy,
oxidising the Group IV metal or alloy in a first oxidation step at a temperature in the range of 500° C. to 900° C. using a carbon containing gaseous species having a first oxidising potential to provide an intermediary Group IV metal oxide,
oxidising the intermediary Group IV metal oxide in a second oxidation step at a temperature in the range of 300° C. to 900° C. using a second gaseous species having a second oxidising potential to provide the protective oxide surface layer, which second oxidising potential is higher than the first oxidising potential.

12. The method according to claim 11, wherein the carbon containing gaseous species is CO₂ or a mixture of CO₂ and CO.

13. The method of claim 12, wherein the carbon containing gaseous species is a mixture of CO₂ and CO, and wherein the ratio of CO₂ to CO and CO₂ is in the range of 0.4 to 0.9.

14. The method according to claim 11, wherein the second gaseous species is N₂O or O₂, or a combination of N₂O and O₂.

15. The method according to claim 11, wherein the first gaseous species and/or the second gaseous species do not comprise hydrogen containing species.

16. The method according to claim 11, wherein the first oxidation step is performed at a temperature in the range of 700° C. to 800° C.

17. The method according to claim 11, wherein the second oxidation step is performed at a temperature in the range of 600° C. to 700° C.

18. The method according to claim 11, wherein the oxidation step has a first reactive duration in the range of 0.2 hour to 24 hours.

19. The method according to claim 11, wherein the second oxidation step has a second reactive duration in the range of 1 hour to 24 hours.

20. The method according to claim 11, wherein the carbon containing gaseous species is CO₂ or a mixture of CO₂ and CO, the first oxidation step temperature is in the range of 700° C. to 800° C., and the first reactive duration is in the range of 12 hours to 24 hours, and the second gaseous species is N₂O in N₂ at a ratio of 20% to 40% N₂O to the total of N₂O in N₂, the second oxidation step temperature is in the range of 600° C. to 700° C., and the second reactive duration is in the range of 12 hours to 24 hours.

21. The method according to claim 11, wherein the first oxidation step is performed with the carbon containing gaseous species at ambient pressure and/or wherein the second oxidation step is performed with the second gaseous species at ambient pressure.

22. The method according to claim 11, wherein the second gaseous species and/or the carbon containing gaseous species is contained in an atmosphere comprising N₂ or an inert gas.

23. The method according to claim 11, wherein the Group IV metal or alloy is selected from the list consisting of titanium, a titanium alloy, zirconium, and a zirconium alloy.

24. The method according to claim 23, wherein the Group IV metal or alloy is titanium grade 1 to 4, Zr702₂, or a titanium/niobium alloy.

25. The method according to claim 11, wherein the Group IV alloy does not comprise aluminium.

26-27. (canceled)
